(19) United States
(12) Patent Application Publication
(10) Pub. No.: US 2004/0219596 A1
(43) Pub. Date: Nov. 4, 2004

(54) Modified carbon nanotubes as molecular labels with application to DNA sequencing

(75) Inventors: Narayan Sundararajan, San Francisco, CA (US); Andrew A. Berlin, San Jose, CA (US); Mineo Yamakawa, Campbell, CA (US); Valluri Rao, Saratoga, CA (US)

Correspondence Address: SCHWEGMAN, LUNDBERG, WOESSNER & KLUTH, P.A., P.O. BOX 2938, MINNEAPOLIS, MN, 55402, US

(73) Assignee: Intel Corporation

(21) Appl. No.: 10/861,644

(22) Filed: Jun. 4, 2004

Related U.S. Application Data
(63) Continuation of application No. 10/067,029, filed on Feb. 4, 2002.

(51) Int. Cl. C12Q 1/68; G01N 33/53; G01N 33/00

(52) U.S. Cl. 435/6; 435/7.1; 436/145

(57) ABSTRACT

A novel device and method for characterization of molecules is provides that improves characterization accuracy by utilizing larger numbers of reactive molecules that are smaller or shorter in chain length for the analysis procedure. Modification of markers such as nanotubes form nanotube assemblies that are easily detected using a number of surface analysis devices such as AFM and STM. The novel method shown using carbon nanotubes to mark a signature on reactive molecules permits a larger distribution and smaller molecule size of reactive molecules used in characterization of a sample molecule. The modification of the carbon nanotubes allows the characterization procedure to detect the nanotube markers more easily, thus decreasing characterization errors, and allowing faster characterization speeds.

This application is a continuation of U.S. patent application Ser. No. 10/067,029, filed on Feb. 4, 2002, which is incorporated herein by reference.

### TECHNICAL FIELD OF THE INVENTION

The present invention relates generally to the field of detection and identification of molecular species. In particular, the present invention relates to identifying and sequencing DNA.

### BACKGROUND

The medical field, among others, is increasingly in need of techniques for identification and characterization of molecules. In particular, techniques for sequencing a DNA molecule have become more important due in part to recent medical advances utilizing genetics and gene therapy.

For a variety of reasons, it has become advantageous to know the sequence of particular DNA molecules. Methods currently exist to map the sequence of DNA, however existing methods are too cumbersome and slow to meet the current characterization and sequencing demands. One such current method includes Automated sequencing machines employing PCR amplification to make many copies of a molecule, followed by chemical (or radioactive) tagging, gel electrophoresis, and statistical computational methods to calculate the original sequence. This method is very time consuming, and not well suited for today's rapid sequencing demands. Additionally the statistical sequencing of PCR determination leaves a margin for error in characterization that is unacceptable.

For short sequences, a hybridization microarray based method is commonly used, employing biochips such as those marketed by Affymetrix. In these “DNA chips,” multiple identical copies are made of detection molecules. The detection molecules consist of specific, short (<100 bases) sequences of DNA that are carefully synthesized such that their sequence is known. By detecting (typically optically) hybridization of the unknown DNA to one of these known short sequences, the sequence of a short portion of the original DNA molecule may be inferred. A problem with the biochip method is that the detection molecules are still too long to provide the accuracy of detection that is desired in the marketplace.

What is needed is a device and method for characterizing molecules that reduces the possibility of characterization errors such as inconclusive readings and misidentified readings. What is also needed is a device and method for characterizing molecules that can be performed at faster speeds.

### BRIEF DESCRIPTION OF THE DRAWINGS

FIG. 1 shows a variety of carbon nanotubes.

FIG. 2 shows a carbon nanotube that has been modified according to the invention.

FIG. 3 shows a disproportionate scale diagram of a reactive molecule according to the invention

FIG. 4A shows a diagram of a reaction chamber according to the invention.

FIG. 4B shows a diagram of a substrate and molecule assemblies according to the invention.

FIG. 4C shows a diagram of one surface analysis device and substrate according to the invention.

FIG. 5 shows a diagram of one possible surface analysis device according to the invention.

FIG. 6 shows a diagram of another possible surface analysis device according to the invention.

### DETAILED DESCRIPTION

In the following detailed description of the invention reference is made to the accompanying drawings which form a part hereof, and in which are shown, by way of illustration, specific embodiments in which the invention may be practiced. In the drawings, like numerals describe substantially similar components throughout the several views. These embodiments are described in sufficient detail to enable those skilled in the art to practice the invention. Other embodiments may be utilized, and structural, logical, and electrical changes may be made, without departing from the scope of the present invention. The following detailed description is, therefore, not to be taken in a limiting sense, and the scope of the present invention is defined only by the appended claims.

In the following descriptions, friction coefficients of materials are discussed. A friction coefficient, by definition, describes forces of interaction between at least two objects or surfaces. A friction coefficient can be described as including both an abrasive component, and an adhesive component. Abrasive friction is defined as primarily a mechanical interaction between two objects. In one example of abrasive friction, resistance to movement at an interface between two objects is generated by asperities on the surfaces of the objects rising past each other or breaking off. In contrast, adhesive friction is defined as primarily a chemical interaction between two objects. A friction coefficient may be determined either by abrasive factors, adhesive factors, or a combination of the two.

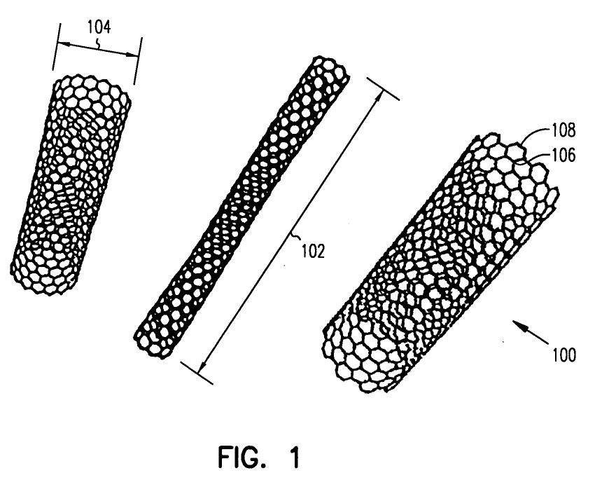

FIG. 1 shows a number of nano-scale fullerene structures 100. Fullerene structures include nanotubes, and spheres that are commonly referred to as buckyballs. FIG. 1 shows a number of carbon nanotubes 100. Carbon nanotubes are nanometer (1×10⁻⁹ meter) sized tube like structures formed from carbon atoms. The nanotubes 100 shown have dimensional variations that distinguish the individual nanotubes 100 from each other. One dimensional variation includes length 102, and another dimensional variation includes diameter 104. Each carbon nanotube 100 includes a number of carbon atoms located at line intersections 106 as diagramed in FIG. 1. Bonds between individual carbon atoms are represented by the lines 108 that are interconnected to form the depicted structure of the carbon nanotubes 100. Further details of the basic structure of a carbon nanotube will be recognized by one skilled in the art.

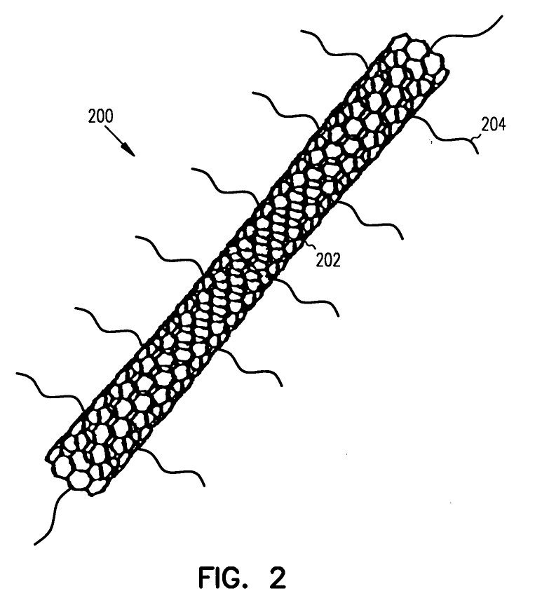

FIG. 2 shows a carbon nanotube assembly 200 that has been modified according to one embodiment of the invention. The nanotube assembly 200 includes a carbon nanotube 202, with a number of additional molecules 204 attached to the nanotube 202 at various locations. The additional molecules 204 are not drawn to scale in the Figure, and the illustration is intended as a diagram to illustrate the modification concept. One skilled in the art will recognize that the number and location of additional molecules 204 can be varied. In one embodiment, several additional molecules 204 are chemically attached to the surface of the carbon nanotube 202 in a homogenous distribution about the surface of the carbon nanotube 202. Although carbon nanotubes are shown in FIG. 2, other fullerene structures such as spheres can be used in alternative embodiments.

The attachment of additional molecules 204 to the surface of the carbon nanotube 202 serves to modify a coefficient of friction of the carbon nanotube 202 with respect to a surface analysis device that will later be discussed in detail. Although the embodiment shown in FIG. 2 shows modification of a surface of the carbon nanotube 202, other embodiments within the scope of the invention include modification the second object forming the friction interface. In one embodiment, the second object includes a component of a surface analysis device, such as a cantilever from an atomic force microscope (AFM) or a scanning tunneling microscope (STM) tip.

The newly formed nanotube assembly 200 will provide a coefficient of friction that is distinguishably different from an unmodified carbon nanotube 202. In one embodiment, the coefficient of friction is modified by changing adhesive friction factors. In one embodiment, the coefficient of friction of the nanotube assembly 200 will be raised higher than the coefficient of friction of the carbon nanotube 202 alone. In another embodiment, the coefficient of friction of the nanotube assembly 200 will be modified lower than the coefficient of friction of the carbon nanotube 202 alone. One skilled in the art will recognize that although the embodiment in FIG. 2 shows additional molecules attached to the carbon nanotube 202 to modify a coefficient of friction, other methods of modifying the coefficient of friction are within the scope of the invention. Other methods may include, but are not limited to, modification of abrasive friction factors such as physical surface modification of the carbon nanotube without attachment of additional molecules.

In one embodiment, the additional molecules 204 attached to the carbon nanotube 202 include carboxylic acid moieties. One method used to attach carboxylic acid moieties to the carbon nanotube 202 includes an acid treatment. The carbon nanotubes 202 are immersed in an acid solution. In one embodiment, the acid immersion takes place at approximately room temperature. Although various acid solutions may be used, in one embodiment, the acid solution includes concentrated sulfuric acid and concentrated nitric acid. The nanotubes are later placed in a mixing device such as an ultrasonicator for a period of time to ensure proper mixing and acid reaction on all surfaces of the nanotubes 202. Any excess acid is distilled off, and the nanotubes are then rinsed in a solution such as ethanol or acetone to rinse away unwanted acid solution. A de-ionized water rinse is performed to further rinse the nanotubes 202. The preceding acid treatment is one method of attaching additional molecules 204 to the surface of nanotubes 202 for modification of an adhesive coefficient of friction. Other methods of molecular attachment or fiction modification may also be used within the scope of the invention.

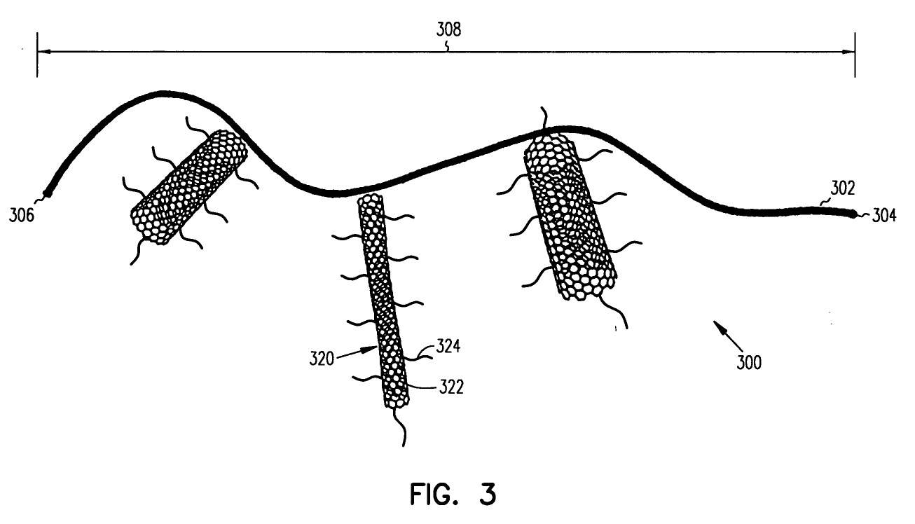

FIG. 3 shows a molecular identification assembly 300. The molecular identification assembly 300 includes a reactive molecule 302. In one embodiment, the reactive molecule includes an assay molecule adapted for hybridization reactions with a long chain sample molecule such as a DNA molecule. Any number of possible reactive molecules are used with the invention. When used for sequencing DNA sample molecules, several thousands of variations of reactive molecules are used. In one embodiment, the variations of reactive molecule include chain molecules, each of approximately 18 monomers in length. Short reactive molecules provide a more detailed characterization of sample molecules being tested.

In the embodiment shown in FIG. 3, the reactive molecule,302 has a first end 304, a second end 306, and a length 308. A number of nanotube assemblies 320 are shown attached along the length 308 of the reactive molecule 302. The nanotube assemblies 320 each include a carbon nanotube 322 and a number of additional molecules 324 attached to the surface of the nanotubes 322. The nanotube assemblies 320 in one embodiment are similar to the nanotube assemblies 200 described in FIG. 2.

Several combinations of nanotube assemblies are possible for attachment to the reactive molecule 302. The number of nanotube assemblies and attachment locations of nanotube assemblies 320 are varied, and the individual physical dimensions of the nanotube assemblies 320 are varied. The variations between individual nanotube assemblies 320, and between combinations of nanotube assemblies 320 associated with each reactive molecule 302 forms a unique signature that is associated with each individual reactive molecule 302. The nanotube assemblies 320 form a type of bar code identity signature that is later detected to identify the reactive molecule 302 that the signature is associated with. Physical dimensions of the nanotube assemblies 320 that are varied include length and diameter.

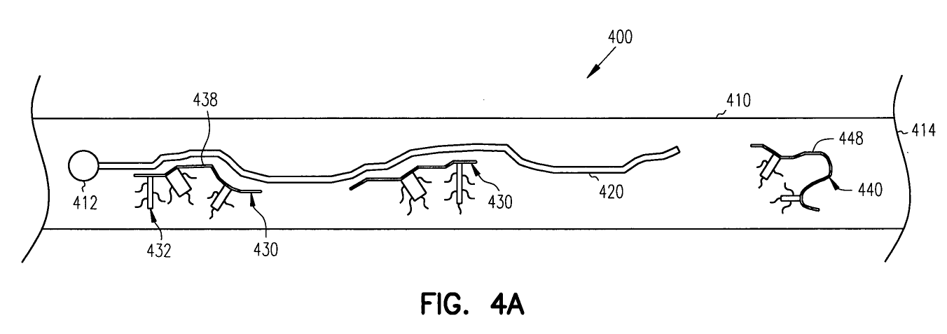

FIG. 4 shows a molecular characterization system 400. The characterization system 400 includes a reaction chamber 410 with an anchor point 412. A sample molecule 420, such as a DNA molecule, is attached at the anchor point 412 in preparation for characterization. A number of molecular identification assemblies 430 are then introduced to the reaction chamber 410 and the sample molecule 420. Each molecular identification assembly 430 includes a reactive molecule 438 with a number of carbon nanotube assemblies 432 attached along a length of the reactive molecule 438. The molecular identification assemblies 430 in one embodiment are similar to the molecular identification assemblies 300 described in FIG. 3. Any number of variations of molecular identification assemblies 430 may be introduced into the reaction chamber 410. In one embodiment, such as a DNA sequencing operation, thousands of variations of molecular identification assemblies 430 are introduced to the reaction chamber 410.

In the characterization process, certain reactive molecules 438 of their associated molecular identification assemblies 430 preferentially associate with, or hybridize with the sample molecule 420. If a known reactive molecule 438 hybridizes at a specific location on the sample molecule 420, an inference can be made about characteristics of the sample molecule, such as the specific sequence of that portion of the sample molecule 420.

In the characterization process, other reactive molecules 448 associated with other molecular identification assemblies 440 will not preferentially associate with the sample molecule 420. These molecular identification assemblies 440 are passed along side the sample molecule 420, and they exit the reaction chamber 410 at a chamber outlet 414.

After the sample molecule 420 has been introduced to a sufficient number of molecular identification assemblies, the sample molecule 420 is removed from the reaction chamber 410 and placed on a substrate 450 as shown in FIG. 4B. The substrate may include, but is not limited to a wafer of silicon, mica, or highly ordered pyrolytic graphite (HOPG). One embodiment includes a patterned substrate that preferentially orients the identification assemblies 430. In one embodiment, the number of molecular identification assemblies 430 that have preferentially associated with the sample molecule 420 are then removed from the sample molecule 420 through a denaturing step. The ordering of the nanotube assemblies 432 along an axis such as 452 is preserved in the denaturing step, and each bar code signature of the reactive molecules may be detected.

In FIG. 4C, a surface analysis device is used to characterize the surface of the substrate 450 and any particles that are on the surface of the substrate such as the number of nanotube assemblies 432. In one embodiment, an atomic force microscope (AFM) is used as the surface analysis device. FIG. 4C shows a portion of an AFM cantilever 470 with an associated tip 472. During the surface analysis of the substrate 450, the tip 472 of the cantilever 470 traces out a scan path 474. As indicated by coordinate axes 460, in one embodiment the scan path includes an x-y scanning plane with scans in the y direction and translations in the x direction. One skilled in the art will recognize that scans in other directions such as the x direction are within the scope of the invention.

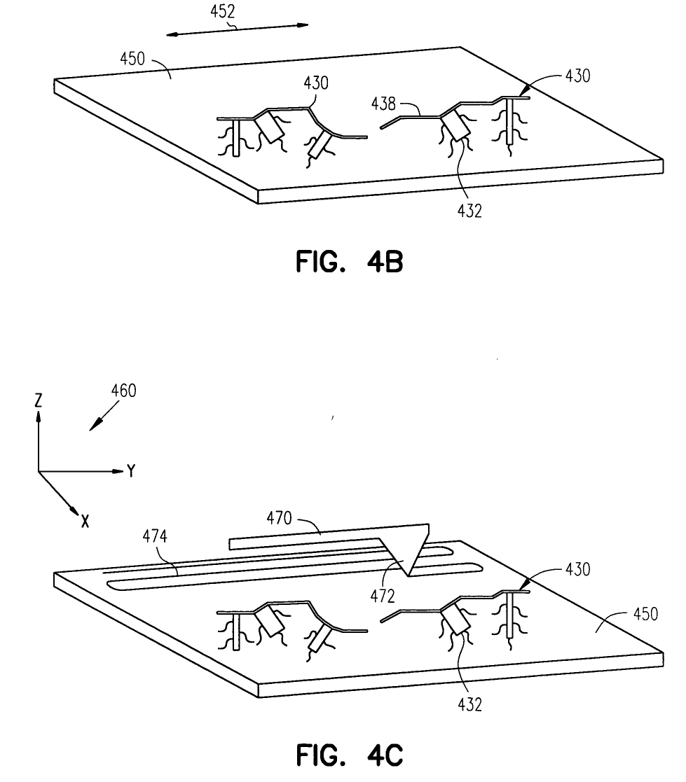

FIG. 5 shows a diagram of selected functional components of an AFM 500 in detail. A cantilever 510 is shown with an arm portion 512 and a tip portion 514. An optical source 520 such as a laser emits a beam 522 toward a backside 515 of the tip portion 514. The beam reflects off the backside 515 and generates a spot 524 on a detector 530. The detector includes a photosensitive plane 532 that detects a two dimensional location of the spot 524 within the photosensitive plane 532. A force 518 acting on the tip portion 514 of the cantilever 510, such as a friction force, causes the tip portion to deflect upwards or downwards along direction 516. The deflection of the tip portion 514 in turn causes movement of the spot 524, which detects the surface characteristics present on a substrate.

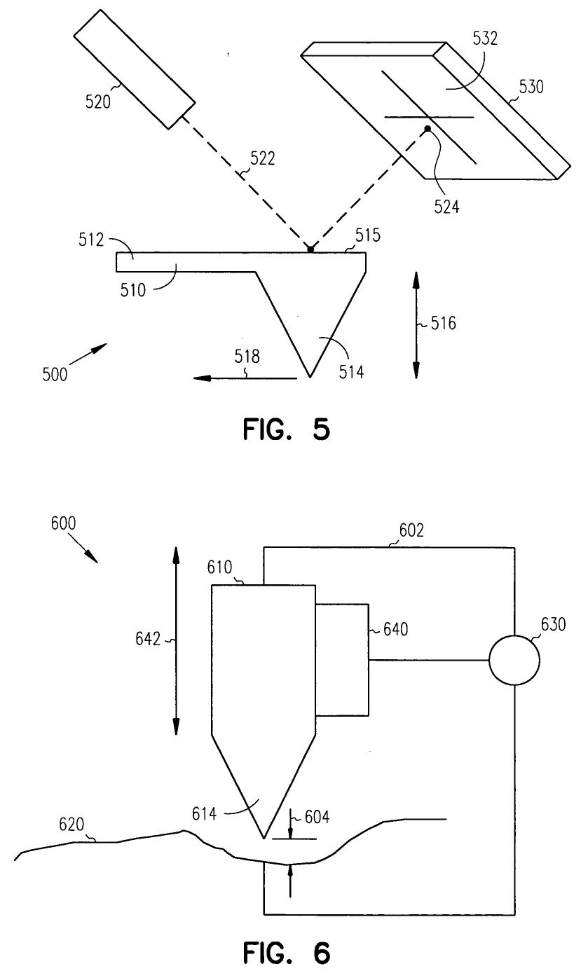

FIG. 6 shows a diagram of selected functional components of a scanning tunneling microscope (STM) in detail. A probe 610, including a tip portion 614 is electrically coupled to the substrate 620 along circuit 602. An electrical characteristic such as an electrical potential is measured between the tip portion 614 and the substrate 620. The electrical characteristic is measured by a detector 630 that provides feedback to a linear actuator 640 such as a piezoelectric device. In one embodiment, a distance 604 between the tip portion 614 and the substrate 620 is monitored and adjusted by a feedback loop. In one embodiment, the actuator 640 is controlled by the detector 630 such that the tip maintains a constant distance 604 over the substrate and the movements of the tip portion record surface characteristics along a given scan line. In another embodiment, a constant height of the tip portion 614 is maintained and variation is an electrical characteristic such as potential are recorded to provide surface characteristics along a given scan line.

By scanning a substrate as prepared in a manner such as shown in FIG. 4C, with a surface analysis device such as an AFM or an STM, a pattern of nanotube assemblies 432 is detected. The pattern of nanotube assemblies indicates a type of a bar code signature of a number of reactive molecules that are associated with the pattern of nanotube assemblies 432. The detected pattern of nanotube assemblies 432 can be related to characteristics of the sample molecule tested, such as a sequence of the sample molecule.

Modification of the carbon nanotubes to create nanotube assemblies 432 as described above alters a friction coefficient at an interface between a first object such as the carbon nanotube assembly, and a second object such as an AFM cantilever tip 472. Modification of the friction coefficient greatly enhances the detectability of the nanotube assemblies 432. The friction coefficient can be raised or lowered depending on the type of additional molecules that are attached to the carbon nanotubes.

One important factor in detection of the nanotube assemblies is not the friction coefficient itself, but the contrasting friction coefficient with the surrounding substrate. If the friction coefficient between the cantilever tip and the substrate is high, then a low coefficient of friction between the cantilever tip and the nanotube assemblies would be desirable to create high contrast. Likewise, if the friction coefficient between the cantilever tip and the substrate is low, then a high coefficient of friction between the cantilever tip and the nanotube assemblies would be desirable.

Modification of the carbon nanotubes to create nanotube assemblies as described above additionally alters electrical properties of the carbon nanotube assembly. Modification of the electrical properties greatly enhances the detectability of the nanotube assemblies to techniques such as STM. Properties such as resistance can be raised or lowered depending on the type of additional molecules that are attached to the carbon nanotubes.

Similar to AFM, an electrical contrast is desirable. If a detected property is high between the STM tip and the substrate, then that electrical property is desirably low in the carbon nanotube assemblies.

### Conclusion

A novel device and method for characterization of molecules has been shown that improves characterization accuracy by utilizing larger numbers of reactive molecules that are smaller or shorter in chain length for the analysis procedure. Modification of markers such as nanotubes form nanotube assemblies that are easily detected using a number of surface analysis devices such as AFM and STM. The method of using carbon nanotubes to mark a signature on reactive molecules permits the larger distribution and smaller molecule size of reactive molecules used in characterization of a sample molecule. The modification of the carbon nanotubes allows the characterization procedure chosen to detect the nanotube markers more easily, thus decreasing characterization errors, and allowing faster characterization speeds.

It is to be understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

We claim:

1. A method of identifying molecules, comprising:
attaching a reactive molecule to a nano-scale fullerene structure on only a first portion of the nano-scale fullerene structure;
modifying a detectable property of the nano-scale fullerene structure on a second portion of the nano-scale fullerene structure, apart from the first portion;
selecting the nano-scale fullerene structure as a result of preferential interaction between the reactive molecule and a sample molecule;
placing the selected nano-scale fullerene structure on a substrate; and
analyzing a surface of the substrate based on the detectable property to detect the nano-scale fullerene structure using a separate measuring device to detect the carbon nanotube.

2. The method of claim 1, wherein the nano-scale fullerene structure includes a carbon nanotube.

3. The method of claim 1, wherein modifying a detectable property includes modifying a friction coefficient.

4. A method of identifying molecules, comprising:
attaching a reactive molecule to a carbon nanotube on only a first portion of the carbon nanotube;
modifying a friction coefficient of the carbon nanotube on a second portion of the carbon nanotube, apart from the first portion;
selecting the carbon nanotube as a result of preferential interaction between the reactive molecule and a sample molecule;
placing the selected carbon nanotube on a substrate; and
measuring friction characteristics of the substrate and the carbon nanotube using a separate friction measuring device to detect the carbon nanotube.

5. The method of claim 4, wherein the sample molecule includes a DNA molecule.

6. The method of claim 4, wherein the reactive molecule includes an assay molecule.

7. The method of claim 4, wherein the operations are performed in the order presented.

8. The method of claim 4, wherein the friction coefficient of the carbon nanotube is modified after the carbon nanotube is attached to the reactive molecule.

9. The method of claim 4, wherein modifying the friction coefficient of the carbon nanotube includes increasing the friction coefficient of the carbon nanotube.

10. The method of claim 4, wherein modifying the friction coefficient of the carbon nanotube includes acid treating the carbon nanotube.

11. The method of claim 4, wherein modifying the friction coefficient of the carbon nanotube includes attaching a chemical species to the surface of the carbon nanotube.

12. The method of claim 11, wherein attaching a chemical species to the surface of the carbon nanotube includes attaching a carboxylic acid group to the surface of the carbon nanotube.

13. The method of claim 4, wherein measuring friction characteristics of the substrate and the carbon nanotube using a separate friction measuring device includes atomic force microscopy (AFM) measurements of the friction characteristics of the substrate and the carbon nanotube.

14. A method of identifying molecules, comprising:
attaching a reactive molecule to a carbon nanotube on only a first portion of the carbon nanotube;
modifying electrical properties of a carbon nanotube on a second portion of the carbon nanotube, apart from the first portion;
selecting the carbon nanotube as a result of preferential interaction between the reactive molecule and a sample molecule;
placing the selected carbon nanotube on a substrate; and
detecting the carbon nanotube using electrical surface detection techniques.

15. The method of claim 14, wherein modifying the electrical properties of the carbon nanotube includes acid treating the carbon nanotube.

16. The method of claim 14, wherein detecting the carbon nanotube includes detecting the carbon nanotube using scanning tunneling microscopy (STM) measurements.

17. The method of claim 14, wherein the sample molecule includes a DNA molecule.

18. A method of forming a molecular identification assembly, comprising:
attaching a reactive molecule to a carbon nanotube on only an end of the carbon nanotube; and
modifying a friction coefficient of side surfaces of the carbon nanotube.

19. The method of claim 18, wherein attaching the reactive molecule to the carbon nanotube includes attaching an assay molecule adapted for combining with portions of a DNA molecule to the carbon nanotube.

20. The method of claim 18, wherein modifying the friction coefficient of the carbon nanotube includes increasing the friction coefficient of the carbon nanotube.

21. The method of claim 18, wherein the operations are performed in the order presented.

22. The method of claim 18, wherein the friction coefficient of the carbon nanotube is modified after the carbon nanotube is attached to the reactive molecule.

23. The method of claim 18, wherein modifying the friction coefficient of the carbon nanotube includes acid treating the carbon nanotube.

24. The method of claim 18, wherein modifying the friction coefficient of the carbon nanotube includes attaching a chemical species to the surface of the carbon nanotube.

25. The method of claim 24, wherein attaching the chemical species to the surface of the carbon nanotube includes attaching a carboxylic acid group to the surface of the carbon nanotube.
